(19) United States
(12) Patent Application Publication
(10) Pub. No.: US 2019/0017088 A1
(43) Pub. Date: Jan. 17, 2019

(54) RADIATION SENSOR

(71) Applicant: Purdue Research Foundation, West Lafayette, IN (US)

(72) Inventors: Babak Ziaie, West Lafayette, IN (US); Chang Yoon, West Lafayette, IN (US)

(21) Appl. No.: 15/878,410

(22) Filed: Jan. 23, 2018

Related U.S. Application Data
(60) Provisional application No. 62/449,307, filed on Jan. 23, 2017.

(51) Int. Cl. C12Q 1/02; G01T 1/16; C12M 1/42; C12M 1/34

(52) U.S. Cl. CPC C12Q 1/02; G01T 1/1606; C12M 41/46; C12M 41/40; C12M 35/02

(57) ABSTRACT

A radiation sensor, comprising a housing, a first chamber disposed in the housing and configured to contain a microorganism. A second chamber is disposed in the housing and configured to contain a fermentation material, the second chamber separated from the first chamber by a breakable separator. A breaking member is configured to break the breakable separator when pressed by a user. A flexible membrane is configured to flex when the microorganism ferments and thereby releases a gaseous byproduct. An electronic indicator is configured to relay information indicating the amount of fermentation, when the radiation sensor has been exposed to radiation less fermentation takes place resulting in a smaller volume of released gaseous byproduct.

### CROSS-REFERENCE TO RELATED APPLICATIONS

The present patent application is related to and claims the priority benefit of U.S. Provisional Patent Application Ser. No. 62/449,307, filed Jan. 23, 2017, the contents of which is hereby incorporated by reference in its entirety into the present disclosure.

### TECHNICAL FIELD

The present application relates to radiation sensors, and specifically to radiation sensors using a biological organism reaction to radiation in order to control a warning mechanism.

### BACKGROUND

This section introduces aspects that may help facilitate a better understanding of the disclosure. Accordingly, these statements are to be read in this light and are not to be understood as admissions about what is or is not prior art.

Many forms of radiation are commonly used for health care, power plants, and food sterilization. Excessive exposure to ionizing radiation can pose health risks that may be detrimental or fatal to those affected; thus, it is important to monitor/measure radiation exposure among individuals who work in high-risk environments (e.g., nuclear plant operators) or are exposed to nuclear accidents (e.g., Fukushima disaster). Nevertheless, even low levels of high-energy radiation exposure (e.g., gamma rays) pose significant health risks (e.g., cancer) for radiation workers. Although high-risk working sites (e.g., hospitals, laboratories, power plants) are typically equipped with large-scale radiation monitoring systems, workers lack precise effective personal monitoring. Wearable personal dosimeters consisting of diodes and solid-state devices are commercially available; however, they are not cost effective and are thus limited to very high-risk industrial usage. Furthermore, the traditional radiation sensors do not directly correlate with biological damage of the ionizing radiation (i.e., DNA damage, mutation, and cell death).

Although many studies have demonstrated the lethal effect of radiation on living matter, no dosimeter up to now has taken advantage of such sensitivity for creating a direct indicator of radiation-induced biological damage.

There is therefore an unmet need for a novel radiation sensor that can be manufactured at ultralow cost and can correlate to damage to biological systems when exposed to radiation.

### BRIEF DESCRIPTION OF THE DRAWINGS

FIG. 1a shows an exploded view of a radiation sensor according to one embodiment.

FIG. 1b shows the radiation sensor of FIG. 1a after assembly.

FIG. 1c shows a cross-sectional view of the radiation sensor of FIG. 1a.

FIG. 2a shows a first step in a manufacturing process of the radiation sensor of FIG. 1a.

FIG. 2b shows a second step in a manufacturing process of the radiation sensor of FIG. 1a.

FIG. 2c shows a third step in a manufacturing process of the radiation sensor of FIG. 1a.

FIG. 2d shows a fourth step in a manufacturing process of the radiation sensor of FIG. 1a.

FIG. 2e shows a fifth step in a manufacturing process of the radiation sensor of FIG. 1a.

FIG. 2f shows a sixth step in a manufacturing process of the radiation sensor of FIG. 1a.

FIG. 3 is a graph which shows measured resistance over time for various radiation levels using the sensor of FIG. 1a.

FIG. 4 is a graph which shows rate of resistance as a function of radiation dose using the sensor of FIG. 1a.

FIG. 5 shows a plan view of a radiation sensor according to one embodiment.

FIG. 6a shows a first step in a manufacturing process of the radiation sensor of FIG. 5.

FIG. 6b shows a second step in a manufacturing process of the radiation sensor of FIG. 5.

FIG. 6c shows a third step in a manufacturing process of the radiation sensor of FIG. 5.

FIG. 6d shows a fourth step in a manufacturing process of the radiation sensor of FIG. 5.

FIG. 6e shows a fifth step in a manufacturing process of the radiation sensor of FIG. 5.

FIG. 6f shows a sixth step in a manufacturing process of the radiation sensor of FIG. 5.

FIG. 6g shows a seventh step in a manufacturing process of the radiation sensor of FIG. 5.

FIG. 7 is a graph which shows yeast gas generation as a function of time for various concentration levels.

FIG. 8 is a graph which shows membrane deflection as a function of CO2 injection rate.

FIG. 9 is a graph which shows the effect of radiation dose on membrane deflection.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

An ultralow cost novel radiation sensor which can correlate to damage to biological systems when exposed to radiation is disclosed herein. In particular, to facilitate personal radiation exposure measurements, in one embodiment a low-cost, film-type, disposable radiation dosimeter that utilizes a micro-organic material (e.g., Saccharomyces Cerevisiae) is disclosed. The use of such micro-organic material (e.g., yeast which genetically is closely homologous to humans) aids in predicting biological damage of the ionizing radiation. A change in resistance can be used to quantitatively measure exposure to radiation.

In another embodiment according to the present disclosure, a novel low-cost wearable radiation sensor is disclosed that combines a micro-electromechanical systems (MEMS) structure with a microorganism (e.g., yeast) which utilizes the dose-response effect of ionizing radiation on the microorganism cells as a biologically relevant surrogate to measure radiation. When exposed to radiation, yeast suffers DNA damage, mutations, and/or death, resulting in their decreased viability and ability to ferment a sugar solution. As a result, the microorganism provides a physical response (i.e., gas generation) to radiation that is biologically significant (DNA damage). The radiation sensor according to the present disclosure provides the microorganism housed in a low cost wearable MEMS structure as a biologically sensitive radiation indicator. The dose response viability of yeast cells and the resulting gas generation in presence of sucrose solution is used to deflect a polydimethylsiloxane (PDMS) membrane and activate a Light emitting diode (LED) indicator. The sensor allows radiation detection with sensitivity in the range of −0.195 mm/decade-rad (1-1000 rad). Alternatively, the deflection can be used to measure a change in capacitance that can be used to quantitatively measure the amount of exposure to radiation.

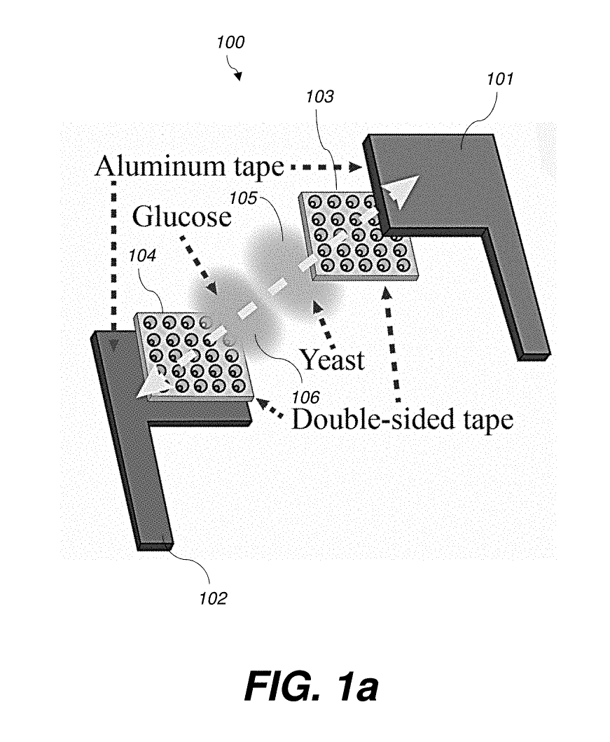

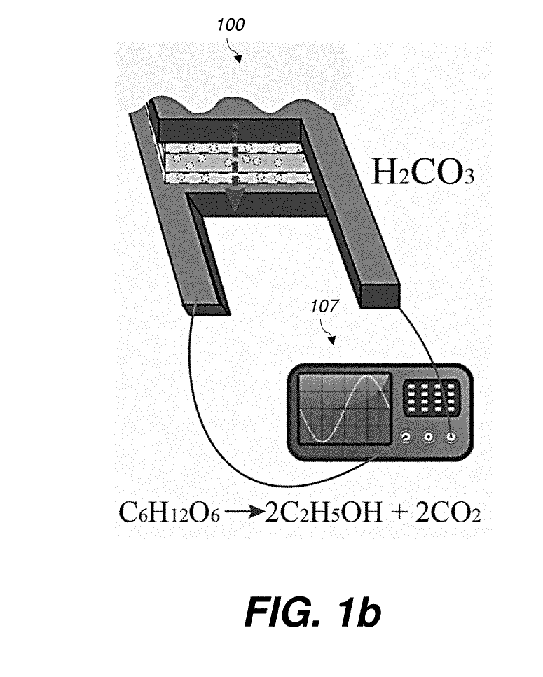

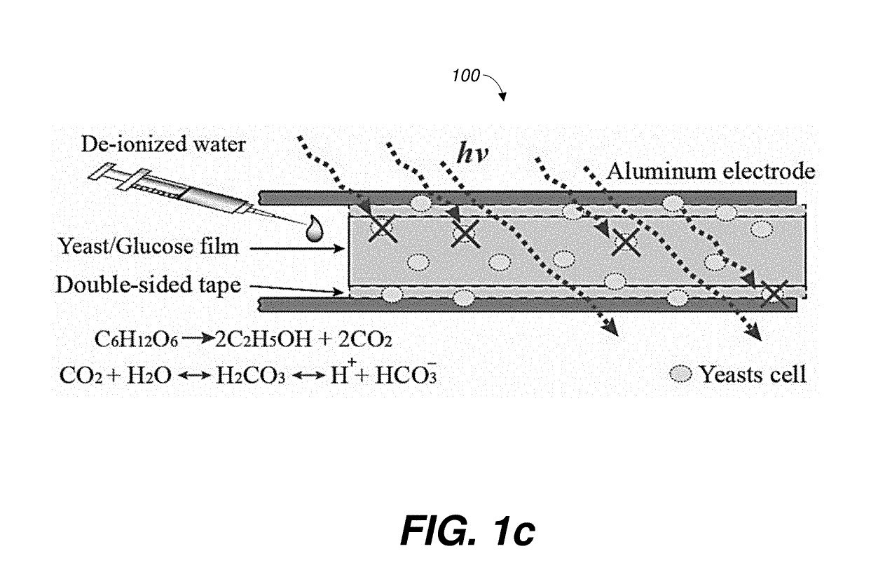

An exploded view of the radiation sensor 100 according to the first embodiment is depicted in FIG. 1(a). The sensor 100 includes two conducting electrodes 101 and 102 (identified as aluminum tape, but could be other electrically conductive material), two layers of adhesive layers 103 and 104 (preferably double-sided adhesive tapes, e.g., SCOTCH double sided tape, made by 3M), a layer of microorganism 105 (e.g., dry yeast; e.g., FLEISCHMANN'S INSTANT DRY), and a layer of fermentation material 106 (e.g., glucose). These layers are provided together as shown in FIG. 1(a), coupled to an electrical meter 107 as shown in FIG. 1(b), and with the cross section of the device 100 as shown in FIG. 1(c), where viable yeast cells (round circles) are fractionally reduced by radiation (as depicted by “X” marks).

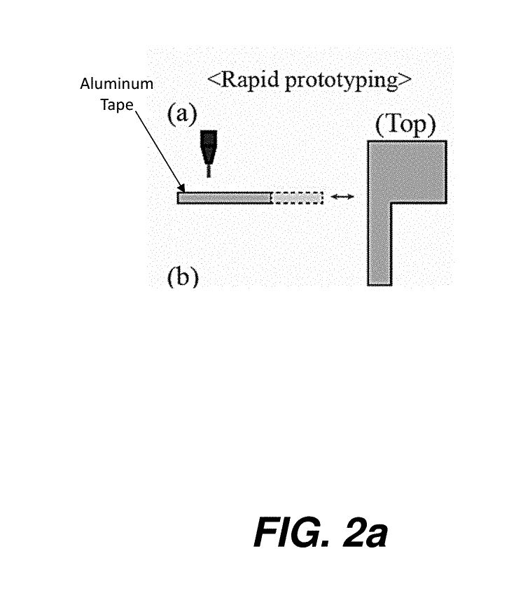

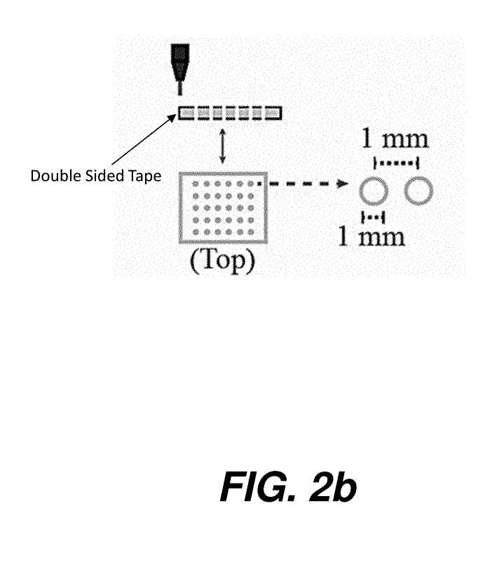

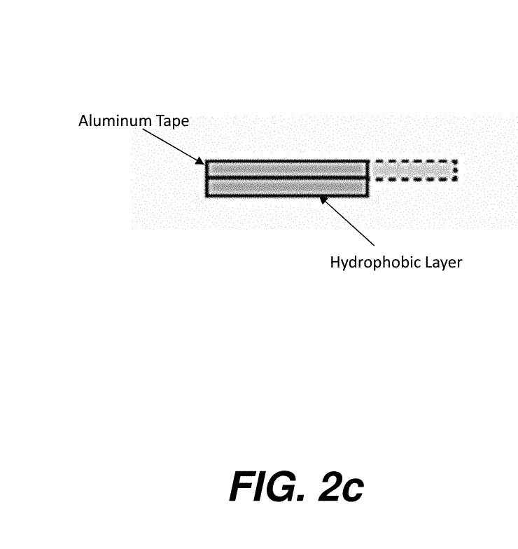

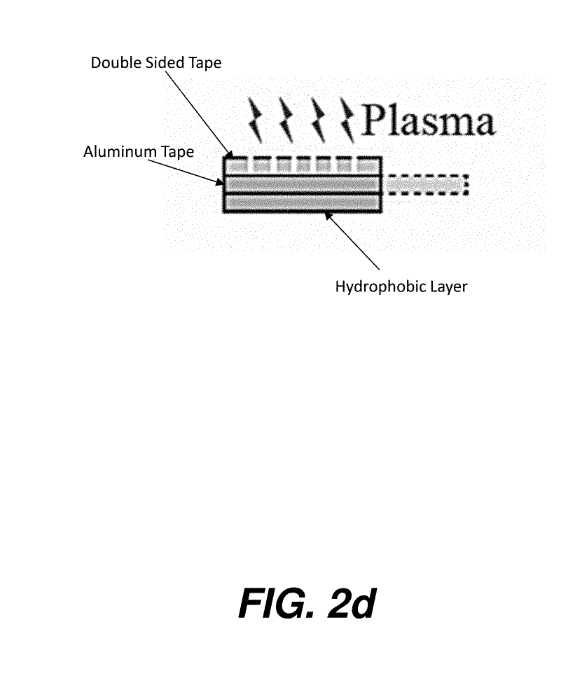

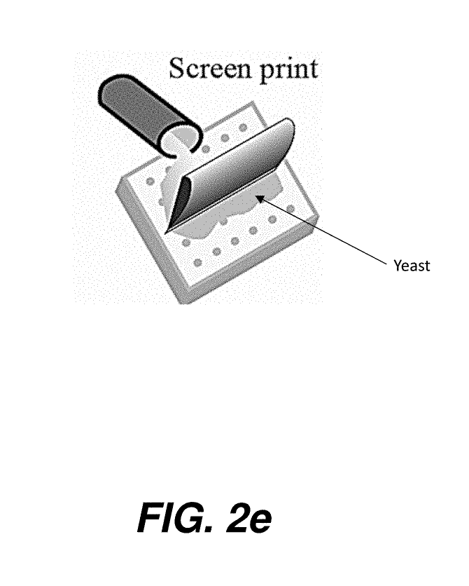

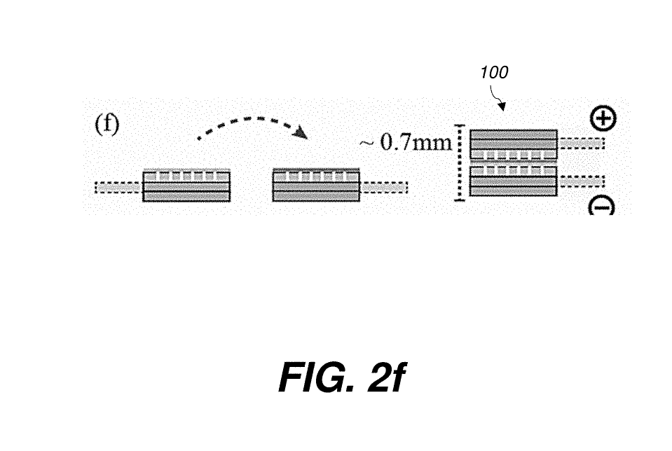

The sensor can be fabricated using rapid prototyping techniques, depicted in FIGS. 2(a)-2(f), according to one embodiment according to the present disclosure. First, two electrically conductive (e.g., metal) electrodes are generated by laser-machining, e.g., aluminum tape into “P” shape (electrode area=10×10 mm²) using a 1.06 μm-wavelength laser engraver system (e.g., PLS6MW, UNIVERSAL LASER SYSTEMS, INC.), as shown in FIG. 2(a). Next, a layer of double-sided adhesive tape (e.g., SCOTCH double sided tape, 3M) is laser machined with a CO₂ laser (10.6 μm) to create a square with a 5×5 array of millimeter-diameter holes (1 mm apart), as shown in FIG. 2(b). The holes provide electrical conduits between aluminum electrodes. To assemble the sensor, each aluminum sheet is sandwiched between a layer of a hydrophobic layer (e.g., freezer paper) and a laser-machined and a double-sided adhesive tape layer, as shown in FIG. 2(c). The tape layers are then plasma-treated to improve hydrophilicity, as shown in FIG. 2(d). After plasma treating, 10 mg of yeast powder (e.g., FLEISCHMANN'S INSTANT DRY) is screen-printed onto the double-sided tape, as shown in FIG. 2(e). The process is repeated on the other tape, but using 10 mg glucose particles (D-(+) Glucose ≥99.5%, e.g., SIGMA-ALDRICH CO.) instead. The tapes are then laminated onto each other, and the sensor is sealed by thermal lamination (380° C. at edges only to avoid damaging yeast) as shown in FIG. 2(f). Fabricated samples are kept refrigerated (4° C.) when not in use to prolong their shelf life.

In operation, ethanol (CH₃CH₂OH) and CO₂ are produced by yeast fermentation when the yeast begins fermenting in presence of glucose. CO₂ reacts with water to form carboxylic acid (H₂CO₃). The generated acid serves as an electrolyte, which alters the electrical conductivity of the solution, which is measured over time. To evaluate the effect of radiation on the electrical properties of yeast, various sensors were exposed to different doses of radiation (0, 10, 100 and 1000 rad) using a Co-60 (1.13 MeV) source. After exposure, each sensor was provided with 0.1 mL of de-ionized water (e.g., using a 30 G hypodermic needle or based on a wicking principal); this sets the yeast concentration inside the sensor to 100 g/L. The sensors were then connected directly to an LCR meter (e.g., LCR-821, GW INSTEK), and the electrical conductivity was measured over time (60 min) at a frequency of 1 kHz, as shown in FIG. 1(b). The measuring frequency of 1 kHz was calculated based on the theoretical capacitance of the film radiation sensor (dry, in air).

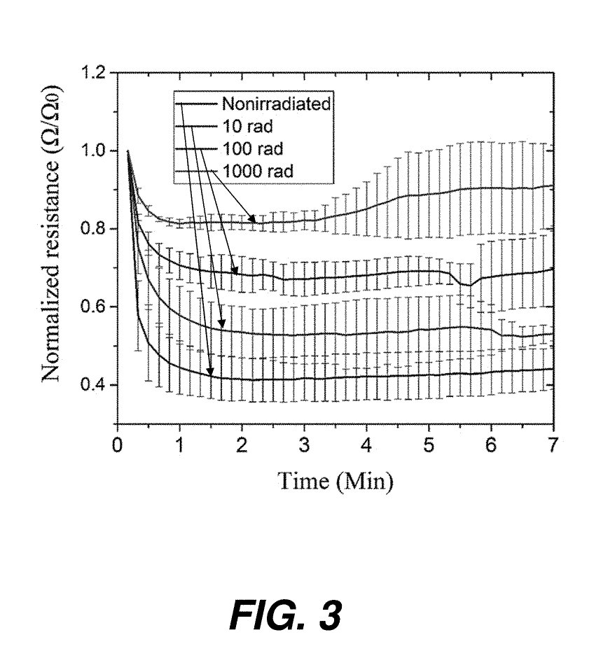

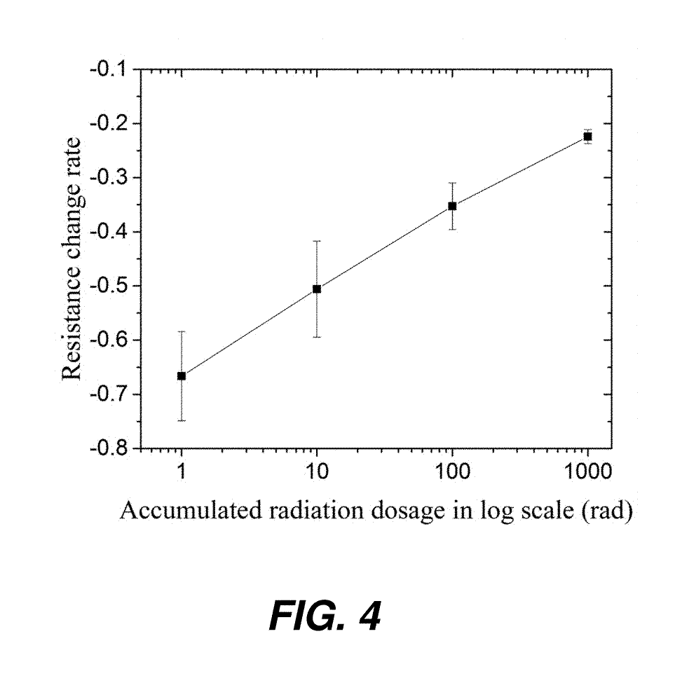

In order to begin the fermentation process, deionized (DI) water is added to the sensor, by a wicking process or by injecting DI water into the sensor using a hypodermic needle. Once all DI water was evaporated or consumed after an hour, the capacitance reading is infinite, similar to the beginning of the experiment, and the resistance is at its maximum value. The measurement data as shown in FIG. 3 show two operational regimes. The first (0-2 minutes) exhibits a drastic drop in resistance as fermentation initiation generates many carboxylic groups. The second regime shows resistance increasing linearly with time as the ions are consumed. The rate of change of resistance is also a function of radiation dose, as shown in FIG. 4. The sensors show a linear resistivity rate with increasing radiation dosage until 1 minute, with resistivity rate of −0.667 Ω/Ω₀min, −0.506 Ω/Ω₀min, −0.353 Ω/Ω₀min, and −0.224 Ω/Ω₀min for the radiation 0, 10, 100, and 1000 rad, respectively; the average sensitivity of the sensor is 0.142 Ω/Ω₀decade-rad at 1 minute after testing, as shown in FIG. 4.

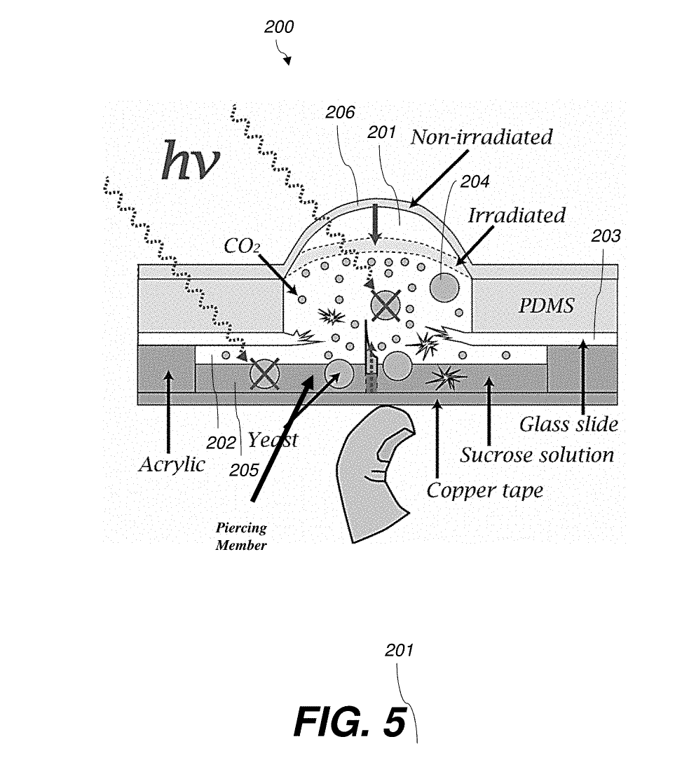

The second embodiment of the radiation sensor according to the present disclosure is based on deformation of flexible membranes that can provide both a pass/fail as well as quantitative results. A plan view of the radiation sensor 200 according to the second embodiment is shown in FIG. 5. The sensor 200 comprises primarily of two chambers 201 and 202 separated by a thin breakable glass separator 203. The upper chamber 201 houses a small colony of powdered yeast cells 204 and the bottom chamber contains an aqueous sucrose solution 205. A commercial dry yeast (FLEISCHMANN'S INSTANT DRY) can be used to increase the shelf life of the device 200; in typical refrigerator conditions, dry yeast can survive for up to 6 months. The sucrose solution 205 serves as a disaccharide nutrition source for fermenting yeast; in a yeast solution, sucrose is hydrolyzed into glucose and fructose (accelerated by the invertase enzyme from yeast). The resulting glucose is then readily consumed by yeast via its enzyme zymase, as shown in the reaction equation provided below:

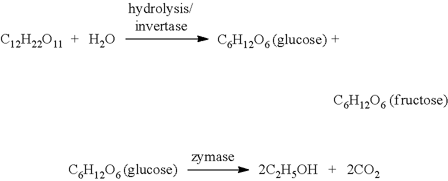

The fermentation process produces carbon dioxide and ethanol. The rate of CO₂ gas generation during fermentation correlates with the activity of the yeast population, which is impaired by radiation exposure; thus gas generation rate is indicative of the radiation dose.

During typical use of the sensor 200, the user wears it during radiation exposure. After exposure, the user breaks the thin glass separator 203 by pressing the back of the sucrose chamber 202, thus mixing the yeast cells 204 with the sucrose solution 205. The resulting fermentation produces CO₂ that can deflects a PDMS membrane 206 (dashed line indicating deflected state). In the absence of radiation, the generated CO₂ is sufficiently large to deflect the membrane (solid line) enough in order to close a switch and turn on an LED indicator. If radiation exposure is large enough to deactivate a significant number of yeast cells, the diminished CO₂ byproduct cannot turn on the LED. Thus, this sensor 200 translates the irradiation-induced biological damage of yeast to a visual LED indicator.

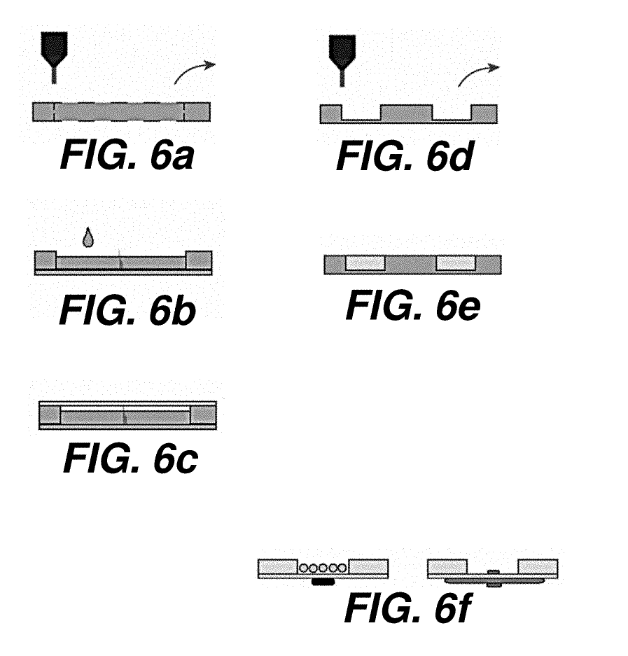

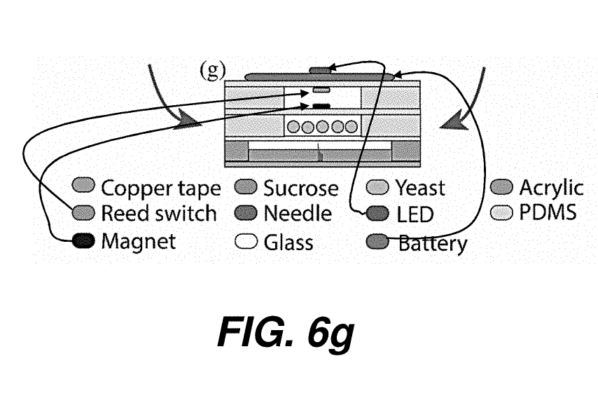

One fabrication embodiment is shown in FIGS. 6(a)-6(f). The radiation sensor 200 includes three components with fabrication sequences as follows: a sucrose solution reservoir (FIGS. 6(a)-6(c)), a yeast chamber, and a LED indicator (FIGS. 6(d)-6(f)). All chambers and other components may be formed by rapid prototyping techniques (e.g., laser machining and layer-by-layer assembly).

The sucrose reservoir is fabricated using impermeable materials to prevent evaporation. First, an acrylic ring (ID 15 mm, OD 20 mm, thickness 5.6 mm) is laser cut using CO₂ laser engraver system (e.g., PLS6MW, UNIVERSAL LASER SYSTEMS, INC.), as shown in FIG. 6(a). A copper disc (20 mm diameter, 100 μm thick) is cut from copper tape using the same system but with a 1.06 μm-wavelength laser. The acrylic ring is then bonded to the copper disc with UV-curable adhesive (LOCTITE 3105) to provide a chamber. Next, a sharp needle tip is glued to the center of the flexible copper tape to assist with breaking the glass or sensor read-out, as shown in FIG. 6(b). 200 μL of a 50 mM sucrose solution (de-ionized water and sucrose) is poured into the chamber, and a glass strip (e.g., a microscope glass coverslip (e.g., TED PELLA, INC., thickness 0.2 mm)) is bonded over the acrylic ring with two-part epoxy, as shown in FIG. 6(c). The sealed chamber is watertight and prevents evaporation of the water from the sucrose solution.

Two PDMS rings are provided using PDMS cast on a laser-machined acrylic mold (8 mm inner diameter, 15 mm outer diameter, 5.6 mm height), as shown in FIGS. 6(d)-6(e). A 200 μm PDMS membrane is created by spin-coating 3 mg of PDMS prepolymer on a silanized silicon wafer (500 rmp, 30 s) and curing it at 80° C. for one hour. A small permanent magnet (K&J MAGNETICS, INC. diameter=3 mm, height=1 mm) is glued to the membrane, and the membrane is then removed from the wafer and bonded to one of the rings using oxygen plasma, forming a chamber. This chamber is subsequently loaded with 20 mg of yeast, as shown in FIG. 6(f) (left panel).

The other PDMS ring is bonded to a 5 mm-thick PDMS substrate embedded with a circuit connecting a reed switch (ORD311, STANDEX-MEDER ELECTRONICS), a battery (3V, CR 1216, RADIOSHACK), and a red LED (VISUAL COMMUNICATIONS COMPANY) in series, as shown in FIG. 6(f) (right panel). The two chambers are then stacked and bonded on top of the glass coverslip to complete the fabrication process, as shown in FIG. 6(g). Samples were kept in refrigerated condition (5° C.) until testing time to prevent any fermentation caused by permeation of ambient gas through PDMS.

To determine the efficacy of the radiation sensor, the fermentation kinetics of yeast were initially investigated by measuring the gas generation rate at various yeast concentrations (10 g·L⁻¹, 25 g·L⁻¹, 50 g·L⁻¹, and 100 g·L⁻¹). Each yeast sample was placed in a flask with a 50 mM aqueous solution of sucrose and heated at 32° C. (typical human skin temperature) for 30 minutes. The generated CO₂ was collected and measured via a standard pneumatic trough setup.

The deflection of the 200 μm PDMS membrane in the radiation sensor in response to pressurized CO₂ was investigated by injecting CO₂ gas into the PDMS/yeast chamber of the sensor using a 30 G hypodermic needle. Since PDMS is partially permeable to CO₂, the membrane deflection saturates after some time for a given gas flowrate (for sufficiently low flowrates). Experiments determined that the membrane bursts when exposed to flow rates greater than 6-7 mL/min. The maximum deflection of PDMS for various flow rates of CO₂ in the range 0-5 mL/min was measured and recorded. The results of these experiments and the fermentation characterization were used to select a yeast concentration (100 g·L⁻¹) for use in the sensor for optimum membrane deflection.

The effect of radiation on yeast activity was studied by evaluating their gas generation rate after radiation exposure. Yeast samples were exposed to various doses (0-1 krad) of radiation using a Co-60 (1.13 MeV) source. The yeast were then incorporated into the sensors as described in the fabrication procedure (using 100 g·L⁻¹), and the sensors were activated. The resulting maximum deflection of the PDMS membrane was then measured and recorded.

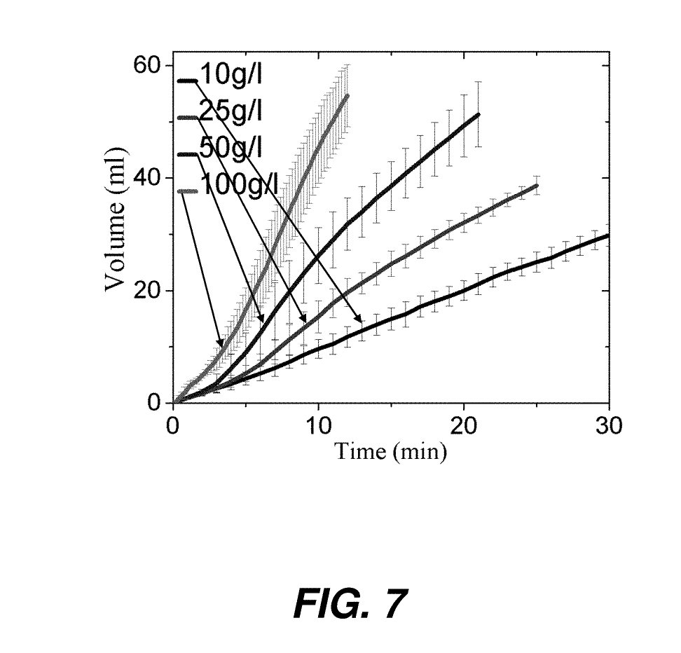

FIG. 7 shows the results of the (non-irradiated) yeast fermentation characterization. For each tested yeast concentration, the CO₂ generation rate increases linearly with time, with rates of 0.8332 ml/min, 1.6251 ml/min, 2.6495 ml/min, and 4.9157 ml/min for the concentrations of 10 g·L⁻¹, 25 g·L⁻¹, 50 g·L⁻¹, and 100 g·L⁻¹, respectively.

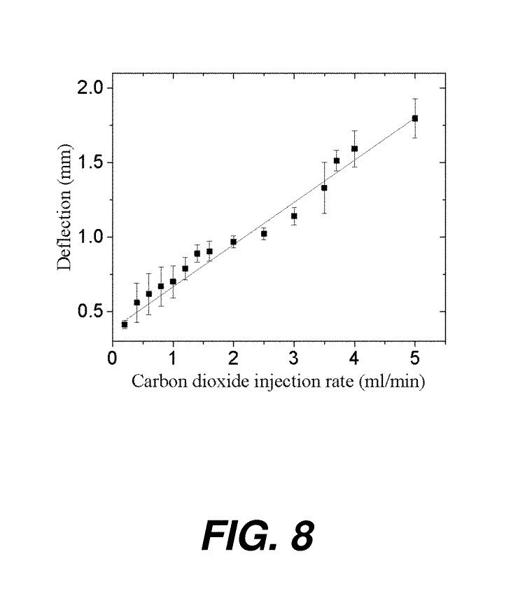

The results of the membrane deflection investigations are plotted in FIG. 8. The data show a linear positive trend between maximum PDMS deflection and CO₂ injection rate for flowrates in the range of 0-5 mL/min and do not burst the membrane. The largest deflection is achieved with 5 mL/min, which is close to the generation rate of a 100 g·L⁻¹ yeast sample; thus 100 g·L⁻¹ was the concentration used when incorporating the yeast into the sensors.

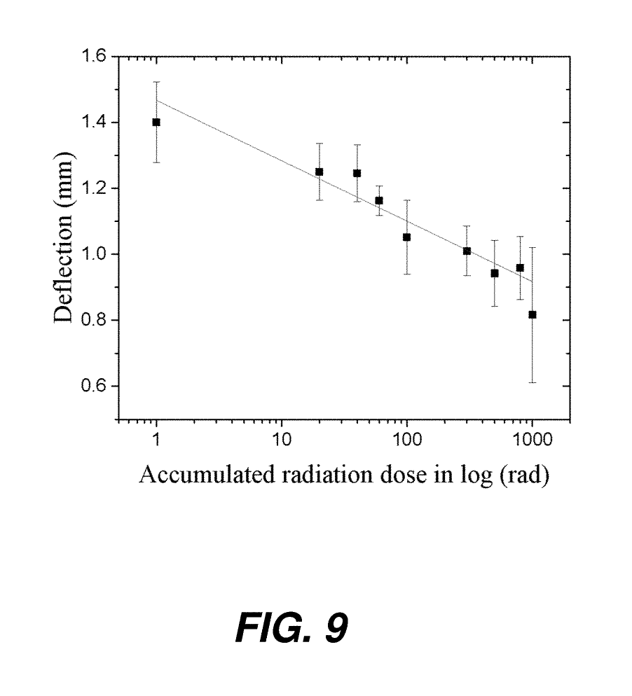

The effect of radiation dose on PDMS membrane deflection is shown in the semi-log plot of FIG. 9. The data suggest an exponential decrease in maximum deflection in response to increasing radiation doses, with an average sensitivity of −0.195 mm/decade of radiation dose (1-1000 rad). The data reveal a sensitivity of −0.00349 mm/rad for doses of 0-100 rad and a lower sensitivity (−0.00026 mm/rad) for doses above 100 rad. Hence, the radiation dosimeter is 13.32 times more sensitive for low doses (1-100 rad) than for higher ones (>100 rad).

Using the radiation and deflection results, the CO₂ generation rate inside the sensor can be back-calculated for each radiation dose using a linear equation, W=0.2728Q+0.4315 (W=Deflection and Q=gas generation rate), obtained from a linear fit of the data in FIG. 8. The CO₂ generation rate can then be used to estimate the metabolic activity of irradiated yeast as a fraction of their non-irradiated counterparts (Table 1). This result shows that on average almost half of the yeast remain unimpaired by radiation doses of up to 500 rad.

TABLE 1
Average percentage of yeast resistance to various radiation doses.

| Radiation | Deflection | CO₂ rate | Remaining |
|---|---|---|---|
| dose (rad) | (mm) | (ml/min) | activity (%) |
| 0 | 1.40 | 3.55 | 100 |
| 20 | 1.25 | 3.00 | 84.50 |
| 40 | 1.245 | 2.98 | 84.02 |
| 60 | 1.162 | 2.68 | 75.40 |
| 100 | 1.051 | 2.27 | 64.01 |
| 300 | 1.01 | 2.12 | 59.7 |
| 500 | 0.942 | 1.87 | 52.70 |
| 1000 | 0.816 | 1.41 | 39.69 |

Those skilled in the art will recognize that numerous modifications can be made to the specific implementations described above. The implementations should not be limited to the particular limitations described. Other implementations may be possible.

1. A radiation sensor, comprising:
a housing;
a first chamber disposed in the housing and configured to contain a microorganism;
a second chamber disposed in the housing and configured to contain a fermentation material, the second chamber separated from the first chamber by a breakable separator;
a breaking member configured to break the breakable separator when pressed by a user;
a flexible membrane configured to flex when the microorganism ferments and thereby releases a gaseous byproduct;
an electronic indicator configured to relay information indicating the amount of fermentation, when the radiation sensor has been exposed to radiation less fermentation takes place resulting in a smaller volume of released gaseous byproduct.

2. The radiation sensor of claim 1, the microorganism is yeast.

3. The radiation sensor of claim 1, the fermentation material is glucose.

4. The radiation sensor of claim 1, the gaseous byproduct is CO₂.

5. The radiation sensor of claim 1, the electronic indicator includes a proximity switch, a battery and a light emitting diode (LED), such that sufficient release of the gaseous byproduct results in closing of the proximity switch and thereby coupling of the battery to the LED and thereby activating the LED.

6. The radiation sensor of claim 1, the electronic indicator includes:
a capacitor formed between the flexible membrane and a non-flexible surface of the housing; and
a capacitance measuring system configured to measure capacitance of the capacitor, the measured capacitance is correlated with the flexure of the flexible membrane which in turn is correlated with the amount of radiation received by the microorganism.

7. A radiation sensor, comprising:
a first conductive electrode;
a second conductive electrode;
a first permeable carrier disposed on the first conductive electrode, the first permeable carrier configured to carry a microorganism; and
a second permeable carrier disposed on the second conductive electrode, the second permeable carrier configured to carry a fermentation material,
the first conductive electrode-first permeable carrier and the second conductive electrode-second permeable carrier are coupled together once the microorganism and the fermentation material are placed on the first and second carriers, respectively, such that when water is added ions form thereby reducing the electrical resistance across the first and second conductive electrodes.

8. The radiation sensor of claim 7, the microorganism is yeast.

9. The radiation sensor of claim 7, the fermentation material is glucose.

10. The radiation sensor of claim 7, the ions are H⁺ and HCO₃⁻.

11. The radiation sensor of claim 7, the first and second conductive electrodes are configured to be coupled to an electrical resistance reader to measure the resistance across the first and second conductive electrodes, where such resistance can be correlated to an amount of radiation received by the microorganism.
